(19) United States
(12) Patent Application Publication
(10) Pub. No.: US 2009/0208545 A1
(43) Pub. Date: Aug. 20, 2009

(54) SYSTEM AND METHOD FOR PROVIDING MEDICINAL TREATMENT

(76) Inventors: Nicole B. Carter-Lyde, Phoenix, AZ (US); Talon J. WhiteOwl, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 12/034,437

(22) Filed: Feb. 20, 2008

(51) Int. Cl. A61K 9/00; A61K 36/28; A61P 17/02; A61K 36/00

(52) U.S. Cl. 424/402; 424/764; 424/725

(57) ABSTRACT

A medicinal delivery device, system and method for providing medicinal treatment to an area skin. The medicinal delivery device may be configured to be a size and shape suitable to conform to the contours of the body (e.g., the perineum region) and configured to retain the medicant, while allowing transfer of the medicinal properties to the skin. The medicinal delivery system may further comprise a containment structure to contain the medicinal delivery device, hold it in place, provide additional comfort, absorb excess water and/or prevent leakage. The medicinal delivery system may further comprise an activation facilitation device to facilitate preparation of the medicinal delivery device for use.

### FIELD OF THE INVENTION

The invention generally relates to medicinal treatments, and more particularly, to systems and methods for providing herbal treatment of skin.

### BACKGROUND OF THE INVENTION

During the process of child birth, stress on the vagina, perineum and rectum may occur resulting in rashes, tears, cutting and/or lacerations. Even if no cutting or tearing occurs during the process, the area may be swollen, bruised or tender. As a result, these areas can feel quite painful. Healing times vary from woman to woman, and discomfort may last for three months or longer.

It is generally known in the art to externally apply various medicants to the affected area to relieve pain and discomfort and promote healing. For example, it is known to soak the affected area in a bathtub of warm water to relieve discomfort. A disadvantage of this technique is that the user must undress in order to immerse the injured area and remain undressed for the duration of the treatment, which may take 20-30 minutes, 3-4 times a day. As such, this technique may be inconvenient for the user.

It is also known to use a sitz bath to relieve postpartum discomfort. A sitz bath may be used to soak the injured area in a shallow plastic basin that is filled with warm water and is usually positioned over a toilet seat. This method of treatment is more convenient than soaking in a bathtub because it requires less time and water to fill the basin and does not require the user to completely undress. However, this method of treatment still requires the user to partially disrobe during treatment (again, usually 20-30 minutes, 3-4 times per day). Moreover, sitting on the hard plastic of a sitz bath after child birth may cause great discomfort. Certain cremes may exist to apply to the affected areas; however, these cremes may get onto clothing or disappear over a short period of time, so the medicinal impact over time is reduced.

Various external herbal treatments are also known. For example, various compresses are known for treating discomfort. A compress is a cloth (e.g., cotton or flannel) that may be soaked in a hot infusion or decoction of an herb, wrung out and applied to the affected area. Such treatments may be held in place by wrapping with an elastic bandage and securing with tape. Such techniques can be messy and time consuming and may inhibit movement of the user. The treatments may also extend over a greater area than desired. Moreover, this technique usually includes partial disrobement to prevent saturation of the user's clothes. Another disadvantage associated with this technique is that in order to effectively wring out the soaked cloth and apply it to the body, the temperature of the cloth often must be reduced to permit handling, which reduces the efficacy and heat-related benefits of the compress.

As such, a system and method that provide localized treatment of injured tissue without the disadvantages discussed above is greatly needed.

### SUMMARY OF THE INVENTION

The present invention overcomes the disadvantages of the known art by developing an inexpensive, convenient system and method for providing medicinal treatment to an area of skin, particularly the vagina, perineum and/or rectum. A medicant delivery device, system and method for use in skin treatment applications are provided in accordance with various aspects of the present invention.

A medical delivery device is any device that may be configured to contain a medicant, while permitting the medicinal properties of the medicant to transfer to a targeted area of skin. In an exemplary embodiment, a medicinal delivery device may include an inner portion configured to comprise a medicant. In an exemplary embodiment, a medicinal delivery device comprises a substantially arrow-shaped configuration in order to facilitate simultaneous coverage of the anus, perineum and vagina.

The medicant may comprise an herbal composition. For example, Calendula or Marigold (Calendula Officinalis), Comfrey (Symphytum officinale), Witch Hazel (Hamamelis virginiana) and/or Yarrow (achillea millefolium) may be used.

In accordance with another exemplary embodiment, the present invention may further comprise a containment structure having an interior portion in which a medicinal delivery device may be contained. More particularly, the containment structure may comprise one or more flaps, which may be overlapping, to contain a medicinal delivery device and one or more wings and/or an adhesive strip to secure the containment structure to the user's garments.

In accordance with another exemplary embodiment, the present invention may further comprise an activation facilitation device configured to hold a medicinal delivery device while it is being prepared for use. The activation facilitation device may, for substance, inorganic substance, plant, liquid and or the like), while permitting a portion or all of the properties of the substance to transfer to a targeted area of an object (e.g., an area of human skin, an animal's skin, a plant).

The medicinal delivery device may comprise any material suitably configured to permit the passage of a portion or all of the medicinal properties of the medicant, while retaining the substance of the medicant. For example, a medicinal delivery device may comprise cheesecloth, muslin and/or filter paper. It will be appreciated by one skilled in the art that any material that suitably allows at least a portion of the medicinal properties of the medicant to transfer through the medicinal delivery device and onto the skin, while the medicant remains contained in the device, may be used.

A medicinal delivery device of the present invention may be configured to be any desired shape, and in particular, any shape that facilitates delivery of the medicant to the targeted skin. For example, medicinal delivery device may be square, rectangular, circular and/or any other known shape or random shape. In accordance with another exemplary embodiment, medicinal delivery device may be configured to conform to the contours of the body and/or fit onto the targeted skin more comfortably. As illustrated in FIG. 1(a), medicinal delivery device 10 may be configured to have a narrower middle portion 12 to facilitate simultaneous coverage of the anus, perineum and vagina. In accordance with another exemplary embodiment, as shown in FIG. 1(b), medicinal delivery device 10 may comprise a substantially arrow-shaped configuration in order to facilitate placement onto the vaginal, perineal and rectal area for greater comfort, increased mobility and accurate targeting of the injured area.

In accordance with another embodiment, medicinal delivery device may be any size to facilitate comfort and deliver optimal treatment. For example, medicinal delivery device may comprise an approximate length of 5 inches, along with approximate widths of 1.5 inches, ¾ inch, and 1 inch. In another exemplary embodiment, medicinal delivery device may comprise an approximate length of 3 inches, along with approximate widths of 1 inch, ½ inch, and 1 inch. However, it will be appreciated by one skilled in the art that a medicinal delivery device of the present invention may comprise any dimensions suitable to contact and/or provide treatment to a desired area. In accordance with an exemplary embodiment, a medicinal delivery device may have an inner portion configured to comprise a medicant.

In an exemplary embodiment, a medicant may be any composition known to have medicinal properties when contacting the surface of skin. For example, a medicant may example, comprise a substantially similar shape as the medicinal delivery device, although it may be slightly bigger to permit the addition of water.

### BRIEF DESCRIPTION OF THE DRAWINGS

A more complete understanding of the invention may be derived by referring to the detailed description and claims when considered in connection with the Figures, wherein like reference numbers refer to similar elements throughout the Figures, and:

FIGS. 1(a) and 1(b) illustrate exemplary diagrams of a medicinal delivery device, in accordance with an exemplary embodiment of the present invention;

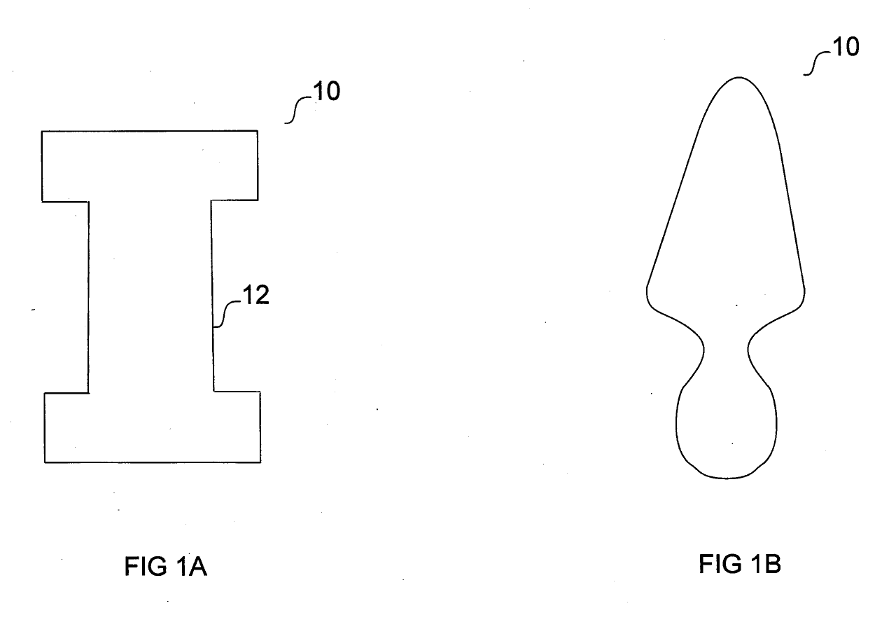

FIGS. 2(a) and 2(b) illustrate exemplary diagrams of configurations of a medicinal delivery system, in accordance with an exemplary embodiment of the present invention; and,

FIGS. 3(a) and 3(b) illustrate exemplary diagrams of an activation facilitation device, in accordance with an exemplary embodiment of the present invention.

### DETAILED DESCRIPTION

The description that follows is not intended to limit the scope, applicability, or configuration of the invention in any way; rather, it is intended to provide a convenient illustration for implementing various embodiments of the invention. As will become apparent, various changes may be made in the function and arrangement of the elements described in these embodiments without departing from the scope of the invention. It should be appreciated that the description herein may be adapted in any number of ways and still fall within the scope of the present invention. Furthermore, different materials, structures, compositions, and the like may be employed in the medicinal treatment systems disclosed herein without departing from the scope of the present invention. Moreover, the various parts and systems herein disclosed may be configured in different arrangements than disclosed while remaining within the scope of the present invention. Thus, the detailed description herein is presented for the purpose of illustration only and not of limitation. References to singular include plural, and references to plural include singular.

In accordance with various exemplary embodiments of the present invention, a medicinal delivery device is provided. While the invention will be described herein in terms of a medicinal delivery device, one skilled in the art will appreciate that the delivery device may contain any substance and deliver any portion of the substance to any other area or object. In an exemplary embodiment, a delivery device is any structure configured to contain any substance (e.g., medicant, comfort material, solution, organic comprise an herbal composition. An herbal composition that may be used in accordance with the present invention may comprise, for example, Calendula or Marigold (Calendula Officinalis), Comfrey (Symphytum officinale), Witch Hazel (Hamamelis virginiana) and/or Yarrow (achillea millefolium). In accordance with an exemplary embodiment, an exemplary composition may comprise 1 parts Calendula, 1 parts Comfrey, 1 parts Witch Hazel, and 1 parts Yarrow.

Desirable properties of a medicant may include, for example, soothing and/or coating of the injured area, rejection of harmful micro-organisms that manifest infection, promotion of healing through proliferation of cell rejuvenation where there is tearing and the like, calming of spasms, and reduction of inflammations, anti-coagulating properties to reduce and prevent bruising and tenderness, astringent properties to reduce bleeding, and properties to improve the immune system and lessen the absorption of toxins. However, it will be appreciated by one of ordinary skill in the art that any medicant, herbal or otherwise, that alleviates, heals or otherwise benefits stressed, stretched, broken, cut and/or irritated tissues, vessels, nerves and/or mucous membranes may be used in accordance with the present invention.

In accordance with another aspect of the present invention, the composition of the medicant may be customized to fit the needs of an individual user. For example, medicinal delivery device may be configured to be preloaded with a medicant composition, or may be configured such that an end-user selects and creates a suitable medicant composition and loads it into the medicinal delivery device.

Once the medicant is inserted, the medicinal delivery device may be sealed using any method suitable to contain the medicant. For example, medicinal delivery device may be sewn shut or sealed using glue or any other adhering and/or adhesive means.

In accordance with another exemplary embodiment, a medicinal delivery device may further comprise a containment structure to form a medicinal delivery system. A containment structure may be any structure configured to contain the medicinal delivery device while permitting the medicinal properties of the medicant contained within to access the targeted tissue.

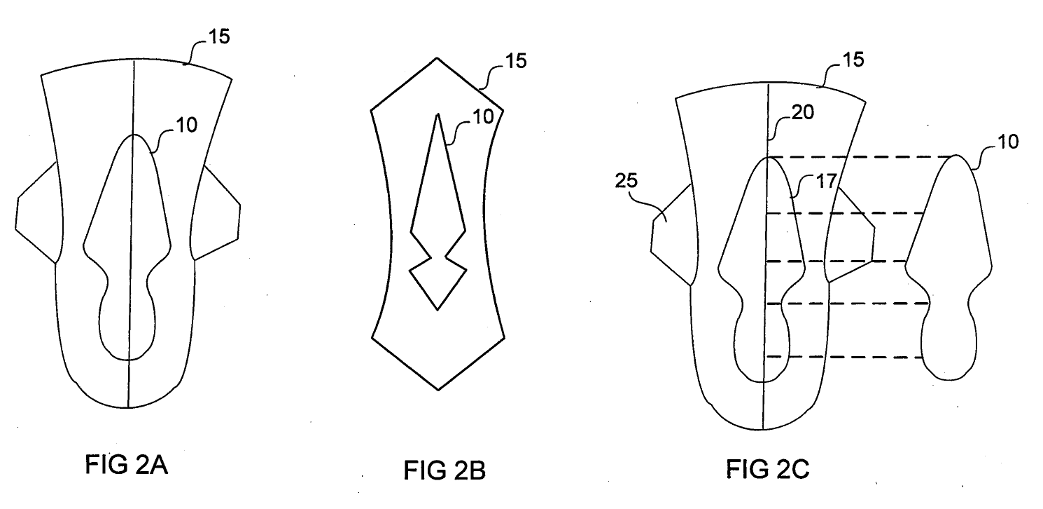

A containment structure according to the present invention may be any size and/or shape to facilitate treatment. In accordance with an exemplary embodiment as shown in FIG. 2A, containment structure 15 may comprise a shape substantially similar, although slightly larger, than a medicinal delivery device 10 to allow a medicinal delivery device to be inserted comfortably inside. As shown in the exemplary embodiment in FIG. 2A, containment structure 15 may be configured to be substantially arrow-shaped.

In accordance with an exemplary embodiment shown in FIGS. 2B and 2C, containment structure 15 may comprise a pocket 17 in which a medicinal delivery device 10 may be inserted. Containment structure 15 may further comprise one or more flaps 20, which may be overlapping, to contain medicinal delivery device 10 once inserted.

In accordance with an exemplary embodiment, the exterior layer of containment structure 15 may be configured to protect against chaffing and/or irritation of targeted skin and facilitate removal of the medicinal delivery system from the skin upon completion of treatment. For example, the exterior layer of containment structure may comprise cotton or synthetic rayon. However, it will be appreciated by one of ordinary skill in the art that any material that allows the medicinal properties of the medicant to reach the skin may be used.

In an exemplary embodiment, medicinal delivery system may be configured to contour to the shape of the body, between sections of the body and/or between body parts in order to naturally hold the medicinal delivery system in place. The containment structure may be configured to fit the natural contour of the area for greater comfort and accurate targeting the injured area.

In accordance with another exemplary embodiment, a containment structure may be configured to provide additional cushioning or padding of the targeted skin area. For example, containment structure may comprise additional layers of cotton, synthetic rayon and/or any other material to create an additional cushioning effect.

In another exemplary embodiment, a containment structure may be configured to absorb excess water emitted by the medicinal delivery device. In this regard, the containment structure may further comprise cellulose fluff or some other material with water-absorbent properties. The cellulose fluff may comprise multiple layers and may be located in the middle of the containment structure so as to soak up any excess water from the medicinal delivery device.

The containment structure of the present invention may also comprise, for example, a moisture-proof material to prevent leakage of excess water. For example, a containment structure may comprise polypropylene or polyethylene.

In accordance with another exemplary embodiment, containment structure may be configured to hold the medicinal delivery device in a desired position. For example, the containment structure may comprise an adhesion mechanism such as an adhesive strip, and one or more wings 25 as shown in FIG. 2C, configured to attach to a garment to hold and support the system in a desired location.

In accordance with another exemplary embodiment, the medicinal properties of the herbal composition may be activated by a solution, such as heated water. In such embodiments, the present invention may further comprise an activation facilitation device.

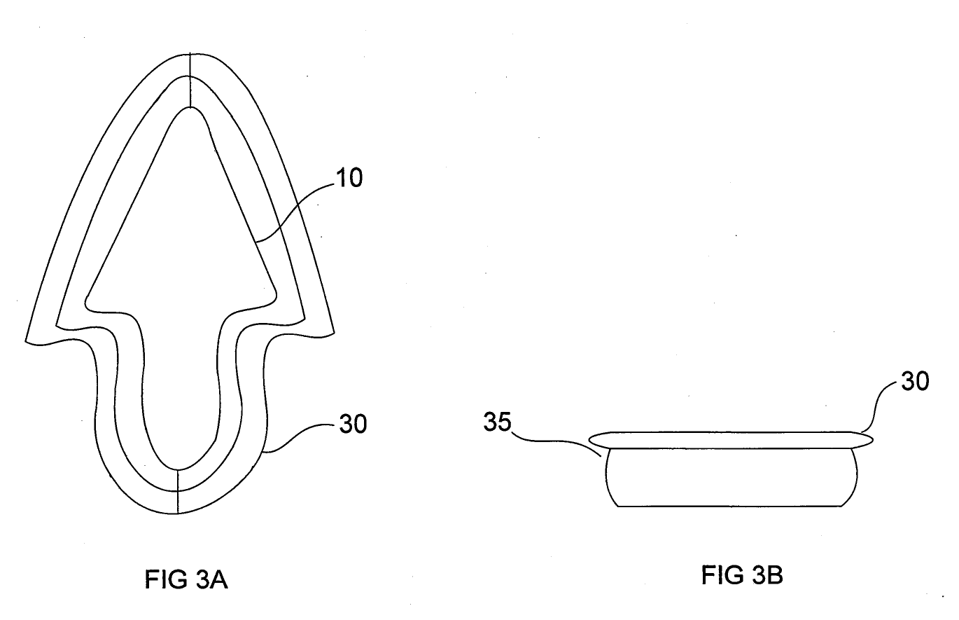

An activation facilitation device may be any device configured to hold medicinal delivery device while it is being prepared for use. For example, as shown in FIGS. 3A and 3B, when a medicant is activated by the addition of hot water, activation facilitation device 30 may be configured to hold medicinal delivery device and hot water. Hot water, preferably boiling, may be poured over medicinal delivery device 10 such that the medicant is allowed to steep.

In accordance with an exemplary embodiment, activation facilitation device may comprise thermoplastic. However, it will be appreciated by one skilled in the art that any material durable enough to withstand water at boiling temperatures without compromising its structural integrity may be used.

It will further be appreciated that activation facilitation device may be any size and/or shape to contain medicinal delivery device. In one exemplary embodiment, as shown in FIG. 3A, activation facilitation device 30 may be comprise a substantially similar shape as medicinal delivery device 10 (e.g., arrow-shaped), only slightly larger to allow the addition of water.

In accordance with another exemplary embodiment, with further reference to FIG. 3A, activation facilitation device 30 may further comprise cover portion 35 to prevent spillage and/or retain the heat of the water. In one exemplary embodiment, cover portion 35 may be configured to cover a portion of the activation facilitation device 30. For example, activation facilitation device may comprise grooves such that the cover portion may be a cover portion onto the top of the activation facilitation device to retain the heat of the water while the medicant steeps. However any suitable method of removably attaching the cover portion to the activation facilitation device may be used.

In accordance with one aspect of the present invention, medicant delivery device is steeped in the activation facilitation device for a suitable length of time. Medicant delivery device may then be removed from the activation facilitation device and inserted into the containment structure to form a medicant delivery system, as depicted in FIG. 2C.

In another exemplary embodiment, the medicine from the herbs may be extracted through a maceration in witch hazel, or any other liquid suitable for extracting the medicinal properties, thus eliminating the need to steep the herbs. In this embodiment, the inner portion of the medicinal delivery device may comprise the maceration composition, for example, in a non-porous, air-tight bag.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the invention. The scope of the invention is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims.

1. A medicinal delivery device comprising:
an interior portion configured to contain a medicant;
an exterior portion having a perimeter;
wherein said exterior portion is configured to permit the passage of the medicinal properties of said medicant while retaining the substance of said medicant;

2. A medicinal delivery device according to claim 1, wherein said perimeter is configured to have a relatively narrow middle portion.

3. A medicinal delivery device according to claim 1, wherein said perimeter is configured to be substantially arrow-shaped.

4. A medicinal delivery device according to claim 1, wherein said external portion comprises at least one of cheesecloth, muslin, and filter paper.

5. A medicinal delivery device according to claim 1, wherein said medicant comprises at least one of Calendula, Comfrey, Witch Hazel and Yarrow.

6. A medicinal delivery device according to claim 1, wherein said medicant comprises Calendula, Comfrey, Witch Hazel and Yarrow present in a ratio of about 1:1:1:1.

7. A medicinal delivery system comprising:
a medicinal delivery device having an interior portion configured to contain a medicant and an exterior portion having a perimeter;
a containment structure having a perimeter and configured to substantially contain said medicinal delivery device;
wherein said exterior portion of said medicinal delivery device is configured to permit the passage of the medicinal properties of said medicant while retaining the substance of said medicant.

8. A medicinal delivery system of claim 7, wherein said perimeter of said containment structure and said medicinal delivery device are configured to be substantially the same shape.

9. A medicinal delivery system of claim 8, wherein said same shape is substantially arrow-shaped.

10. A medicinal delivery system of claim 7, wherein said containment structure comprises at least one of cotton, synthetic rayon and cellulose fluff.

11. A medicinal delivery system of claim 7, wherein said containment structure comprises at least one flap.

12. A medicinal delivery system of claim 7, wherein said containment structure comprises at least one adhesion mechanism.

13. A medicinal delivery system of claim 7, wherein said containment structure comprises a moisture-proof material.

14. A medicinal delivery system of claim 10, wherein cellulose fluff is configured to be substantially in the middle of said containment structure.

15. A medicinal delivery system comprising:
a medicinal delivery device having an interior portion configured to contain a medicant and an exterior portion having a perimeter, said exterior portion configured to permit the passage of medicinal properties of said medicant while retaining the substance of said medicant;
a containment structure having a perimeter, and configured to contain said medicinal delivery device;
an activation facilitation device having a perimeter;
wherein said medicinal properties of said medicant are configured to be activated by placing said medicant delivery device inside said activation facilitation device and adding relatively hot water.

16. A medicinal delivery device according to claim 15, wherein said perimeter of said activation facilitation device, said containment structure and said medicant delivery device are configured to be substantially the same shape.

17. A medicinal delivery device according to claim 16, wherein said substantially same shape is arrow-shaped.

18. A medicinal delivery device according to claim 15, wherein said activation facilitation device comprises thermoplastic.

19. A medicinal delivery device according to claim 15, wherein said activation facilitation device further comprises a cover portion.

20. A medicinal delivery device according to claim 19, said activation facilitation device having grooves to facilitate removable attachment of said cover portion.
